(19) United States
(12) Patent Application Publication
(10) Pub. No.: US 2024/0409997 A1
(43) Pub. Date: Dec. 12, 2024

(54) FLUORESCENT BARCODING OF MICROPARTICLES

(71) Applicant: Celldom, Inc., San Carlos, CA (US)

(72) Inventor: Eugene TKACHENKO, Del Mar, CA (US)

(73) Assignee: Celldom, Inc., San Carlos, CA (US)

(21) Appl. No.: 18/699,868

(22) PCT Filed: Oct. 10, 2022
(86) PCT No.: PCT/US2022/046203

§ 371 Date: Apr. 9, 2024

Related U.S. Application Data
(60) Provisional application No. 63/254,161, filed on Oct. 10, 2021.

(51) Int. Cl. C12Q 1/6874; C12Q 1/6806

(52) U.S. Cl. CPC C12Q 1/6874; C12Q 1/6806

(57) ABSTRACT

The invention provides a method of barcoding microparticles with fluorescent moieties. The fluorescent moieties are linked to the microparticles by variable cleavable linkers. The combination of fluorescent moieties and variable cleavable linkers provides microparticles having an exponentially expandable number of fluorescent barcodes that can be manufactured and decoded in a sequential manner. The fluorescent barcodes can be used to identify microwells, cells, chemicals and oligonucleotides in wide variety of high throughput assay applications.

### CROSS-REFERENCE TO RELATED APPLICATIONS

This application claims priority to U.S. provisional application No. 63/254,161, filed Oct. 10, 2021, the entire contents of which are incorporated herein by reference for all purposes.

### FIELD OF THE INVENTION

The invention generally relates to in vitro assays and methods for their use and manufacture.

### BACKGROUND

Bead encoding is a powerful technique for screening methodologies in biological applications. Fluorescent readouts are standard for image-based screening applications, but the encoding power of fluorophore-labeled beads is only in the hundreds making them of limited use for high-throughput applications. The most common way to encode beads at high encoding power is to generate bead-specific genetic codes. However, identifying these codes is complicated as the codes must be read by sequential hybridization and removal of the DNA oligo probes, or by next generation sequencing.

### SUMMARY OF THE INVENTION

The invention increases the encoding power of beads using fluorophores and cleavable linkers to generate millions of code combinations. The use of fluorophores and cleavable linkers simplifies code identification by eliminating the hybridization, wash, and sequencing steps that are required to resolve genetic based bead codes. Instead, the invention uses simple fluorescent imaging to quickly and efficiently identify up to millions of bead codes.

It is an object of the invention to provide methods and kits for making and detecting fluorescently encoded microparticles.

In some aspects, the invention provides a method of making fluorescently encoded microparticles. The method can be practiced by attaching to microparticles fluorophores, or particles containing different fluorophore(s), using different cleavable linkers. The combination of fluorophores and cleavable linkers on the beads provides the beads with unique barcodes capable of identifying millions of individual beads.

In some aspects, the invention provides a method of identifying, or decoding, fluorescently labeled beads. The method can be practiced by providing beads that are linked (i.e. attached) to various fluorophores of fluorescent moieties using various cleavable linkers. Fluorescent images of the beads are collected, then the cleavable linkers are sequentially treated with an agent that either selectively cleaves the cleavable linkers to release the fluorophores, or activates the fluorophores that are attached to the cleavable linkers. Fluorescent images are taken after the cleaving or activating event to identify the removal or activation of the fluorophores. The images are then compared and subtraction of images before and after each the cleaving or activating event identifies which types of fluorescent particles were released or activated thereby identifying the fluorescent barcodes on the beads. This combinatorial space can be expanded by repeating this process for many cycles of cleaving or activating, and recording the differential fluorescent signal on each bead before and after each cleaving or activating process.

The inventive method can be used to create millions of bead-specific barcodes. Possible applications for the fluorescent bead encoding methods and kits of the invention include fluorescent encoding of bead-specific genetic barcodes and bead-specific chemicals.

The fluorescent bead encoding methods of kits of the invention can be used to improve the data quality of single cell sequencing by excluding data from microwells containing more than one cell. The methods of kits of the invention can also be used in antibody discovery to assign antibody-encoding gene sequences to individual microwells in an array in which antibodies secreted by single B-cells in the microwells and examined using optical readouts.

The fluorescent bead encoding methods of kits of the invention can be used for identification of potent T-cell receptors by enabling the assignment of gene sequences encoding T-cell receptors to individual microwells in which cytotoxicity of single T-cells towards cancer cells is examined using optical readouts.

The fluorescent bead encoding methods of kits of the invention can further be used to assist in identification of molecular targets by CRISPR screening by assigning CRISPR modification in the genome to individual microwells in which functional assays on these cells, such as cell cycle arrest or formation of focal adhesions, is examined using optical readouts.

The fluorescent bead encoding methods of kits of the invention can be used to identify drug resistance mechanisms in cancer cells. Optical readout will be used to visualize clonal expansion of cancer cells exposed to different concentration of drugs, whereas the specific mutations and altered cell signaling will be revealed by single cell sequencing.

The fluorescent bead encoding methods of kits of the invention can be used to investigate the heterogeneity of cell populations by assigning molecular signature revealed by single cell transcriptional or other sequencing-based readouts with live-cell imaging readouts in individual microwells.

### BRIEF DESCRIPTION OF THE DRAWINGS

FIG. 1 is a process diagram showing the decoding of fluorescently encoded beads, wherein “B” refers to blue fluorophores, “Y” refers to yellow fluorophores, “G” refers to green fluorophores, “O” refers to orange fluorophores, and “FR” refers to far-red fluorophores. These references are similarly used throughout this application, including FIG. 2 and Tables 1 and 2.

FIG. 2 is a process diagram showing the encoding of beads with a combination of fluorescent barcodes and DNA barcodes.

FIGS. 3(A)-3(E) are diagrams showing the process for encoding of beads according to Example 1.

### DETAILED DESCRIPTION

The invention provides methods and kits for encoding and identifying (i.e. decoding) fluorescently labeled microparticles. The invention utilizes different fluorophores, or particles containing different fluorophores, which are attached to microparticles by different cleavable linkers which can be selectively cleaved by cleaving agents. Alternatively, the cleavable linkers can be substituted with, or used in combination with, activating linkers that permit the fluorophores (e.g. particles containing the fluorophores) to be activated when treated with an activating agent. Suitable cleavable linkers and activating linkers for use with the invention include, but are not necessarily limited to, DNA, RNA, peptides, and chemical linkers. Cleaving agents and activating agents for use with the invention selectively cleave or activate the cleavable linkers that are selected for linking fluorophores to the microparticles. The cleaving agents and activating agents preferably have sufficient specificity to selectively cleave or activate a targeted linker on a microparticle such that the targeted linker is cleaved or activated without cleaving or activating non-targeted linkers on the microparticle so that the presence of the targeted linker can be detected. Suitable cleaving agents and activating include, but are not necessarily limited to, restriction enzymes, DNAzymes, uracil-specific excision reagent enzymes, proteases, acidic agents, and reducing agents. In some embodiments wherein the linker is an activating linker, the activating linker can link a quencher to a fluorophore such that treating the activating linker with the activating agent selectively releases the quencher from the fluorophore thereby activating the fluorophore so that it fluoresces and can be detected by a suitable means, such as fluorescent microscopy, for example.

In some embodiments, the invention provides kits and methods of providing a library of fluorescently encoded microparticles. The microparticles can be microbeads. The methods can be practiced by ligating, or attaching, different fluorophores (or different fluorophore-containing particles) to microparticles using different cleavable linkers, different activating linkers, or a combination thereof. The many possible combinations of fluorophores, fluorophore-containing particles, cleavable linkers, and activating linkers provide labels which can themselves be combined serve as barcodes capable of specifically identifying individual microparticles. The kits and methods of the invention can be practiced with 1, 2, 3, 4, 5, or more different fluorophores or fluorophore-containing particles. Similarly, the kits and methods can be practiced with 1, 2, 3, 4, 5, or more different cleavable linkers, or 1, 2, 3, 4, 5, or more different activating linkers.

Table 1 shows a non-limiting example wherein twenty-four label combinations are possible using six different colored fluorophores and four different cleavable or activating linkers. These labels can then in turn be used alone, or combined with one another, to provide millions of barcode combinations. For example, using two labels selected from the 24 possible label combinations of Table 1 would yield over 16 million different barcode combinations (2ⁿ−1 where 2²⁴−1=16,777,215). Table 2, demonstrates, for example, that using two labels provided by combining two different fluorophores and two different linkers can provide up to 15 different barcode combinations.

TABLE 1
Linker-Fluorophore Labels

| Linker | Blue | Green | Yellow | Orange | Red | Far Red |
|---|---|---|---|---|---|---|
| 1 | 1B | 1G | 1Y | 1O | 1R | 1FR |
| 2 | 2B | 2G | 2Y | 2O | 2R | 2FR |
| 3 | 3B | 3G | 3Y | 3O | 3R | 3FR |
| 4 | 4B | 4G | 4Y | 4O | 4R | 4FR |

TABLE 2
Barcode Combinations Provided by Four Different Labels

| | | | | | | | |
|---|---|---|---|---|---|---|---|
| 1B | 1G | 2B | 2G | 1B, 1G | 1B, 2B | 1B, 2G | 1B, 1G, 2B |
| 1B, 1G, 2G | 1B, 1G, 2B, 2G | 1B, 2B, 2G | 1G, 2B | 1G, 2G | 1G, 2B, 2G | 2B, 2G | |

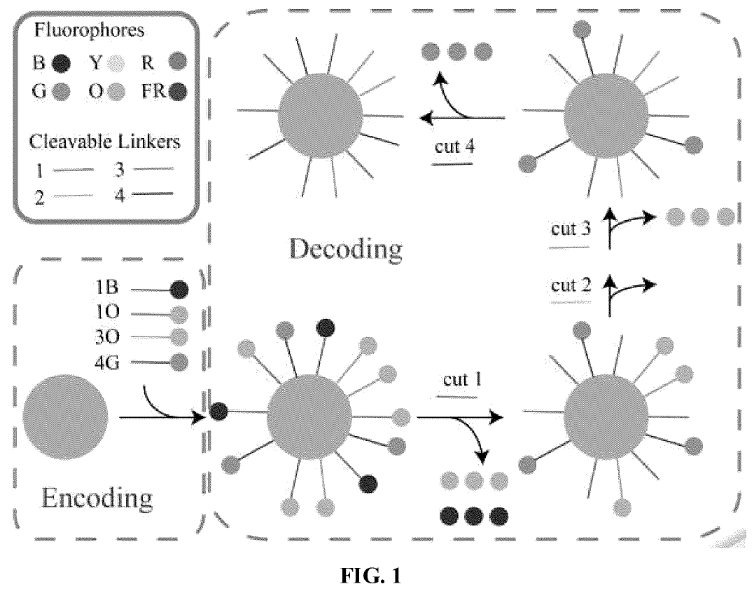

In some embodiments, the invention provides methods of decoding, or identifying, fluorescently encoded microparticles. FIG. 1 shows a process diagram of a non-limiting embodiment of such a method wherein a microparticle labeled with different fluorophore-linker label combinations is subjected to successive cleaving (or cutting) and imaging steps. Performing a first cut using a first agent that selectively cuts a first cleavable linker and releases two different fluorophores from the microparticle. Fluorescent images of the microparticle before and after this cleaving event show that these two fluorophores were linked to the microparticle by the first cleavable linker. Thus, the combination of the selective cleaving agent and fluorescent imaging reveals two labels with the first cutting event. The imaging and selective cleaving of the linkers is then repeated to identify the remaining labels on the microparticle. The identified labels are then combined to identify, or decode, the fluorescent barcode of the microparticle.

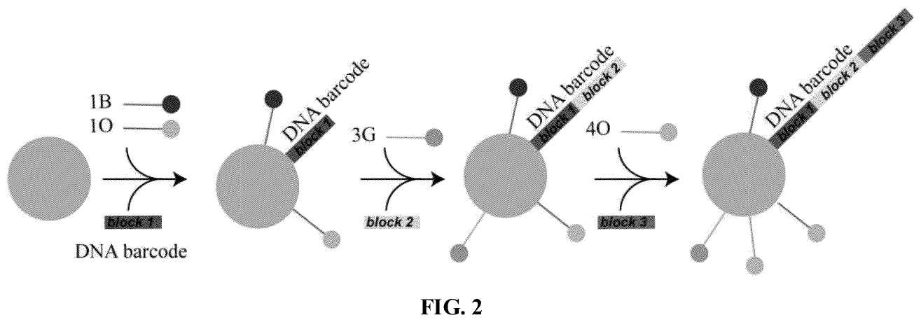

In some embodiments, the invention provides methods and kits for color encoding oligonucleotide barcodes on microparticles. The microparticles can be microbeads (i.e. beads). FIG. 2 is a process diagram showing a non-limiting embodiment of such a method. As shown in the figure, a first linker-fluorophore combination comprising two labels and a first oligonucleotide segment, or block, of a DNA barcode is attached to a microparticle. The first linker-fluorophore combination is associated with the first oligonucleotide segment such that imaging and cleaving of the labels as disclosed herein can detect the presence of the labels and identify the first oligonucleotide segment on the microparticle. Subsequently, two or more oligonucleotide segments are ligated to the first oligonucleotide segment that is attached to the microparticle. Concurrently, two or more linker-fluorophore labels (or label combinations) are attached to the microparticle, wherein the two or more linker-fluorophore labels are associated with the two or more oligonucleotide segments such that the two or more linker-fluorophore labels can be used to identify the presence of the two or more oligonucleotide segments, such as through a lookup table. Accordingly, the combination of linker-fluorophore labels can be used to identify the oligonucleotide segments on the microparticle which combine to provide the DNA barcode.

In some embodiments, the invention provides kits for performing one or more of the methods disclosed herein. The invention can provide kits for making fluorescently encoded microparticles, kits for making libraries of fluorescently encoded microparticles, kits for identifying (i.e. decoding) the barcodes of fluorescently encoded microparticles, and kits for color coding oligonucleotide barcodes on microparticles. The kits can similarly be used in embodiments wherein the microparticles are microbeads (i.e. beads).

The invention described herein is illustrated by the following experiments, which are not be construed as limiting. The contents of any references, pending patent applications and published patents, cited throughout this application are hereby expressly incorporated by reference for all purposes. Those skilled in the art will understand that this invention may be embodied in many different forms and should not be construed as limited to the embodiments set forth herein. Rather, these embodiments are provided so that this disclosure will fully convey the invention to those skilled in the art. Many modifications and other embodiments of the invention will come to mind in one skilled in the art to which this invention pertains having the benefit of the teachings presented in the foregoing description and following examples. Although specific terms are employed, they are used as in the art unless otherwise indicated.

### EXAMPLES

### Example 1

Generation of Fluorescent Barcoded mRNA Capture Beads for Optical Demultiplexing by Multi-Color Fluorescence Imaging for Single Cell RNA-Seq

Basic workflow. The overall goal is to measure phenotypic and transcriptomic information from individual cells using both imaging and sequencing based readouts. Experiments are performed in optically clear microwell arrays, which are fabricated by attaching an embossed microwell array film to the bottom of a 96-well plate frame. Cells are loaded into one or more of the wells of the 96-well plate. The cells sediment into the microwells at the bottom of each well, thus isolating the cells into thousands of discrete microwell-based cell cultures. Standard brightfield and epi-fluorescent imaging is used to collect phenotypic information, such as the number of cells in each microwell, the size and shape of each cell, and also fluorescently labeled molecules that are on the cell surface or in the interior of the cell. In some cases, it is desirable to also quantify cellular secretions in each well by co-assembling beads into the microwells that have capture moieties for different molecules, including but not limited to, nucleic acids, antibodies, cytokines, chemokines, and combinations thereof. The molecules captured on the bead are measured using image- or sequencing-based readouts. High throughput multi-channel imaging is used to identify and assign a unique spatial index to each microwell, which is used for tracking each microwell during time lapse imaging.

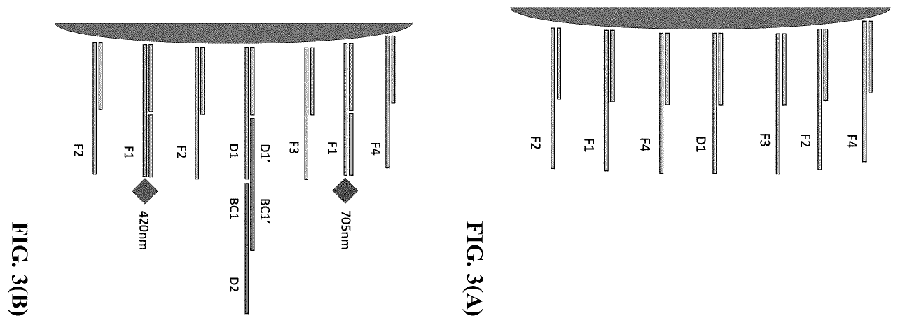

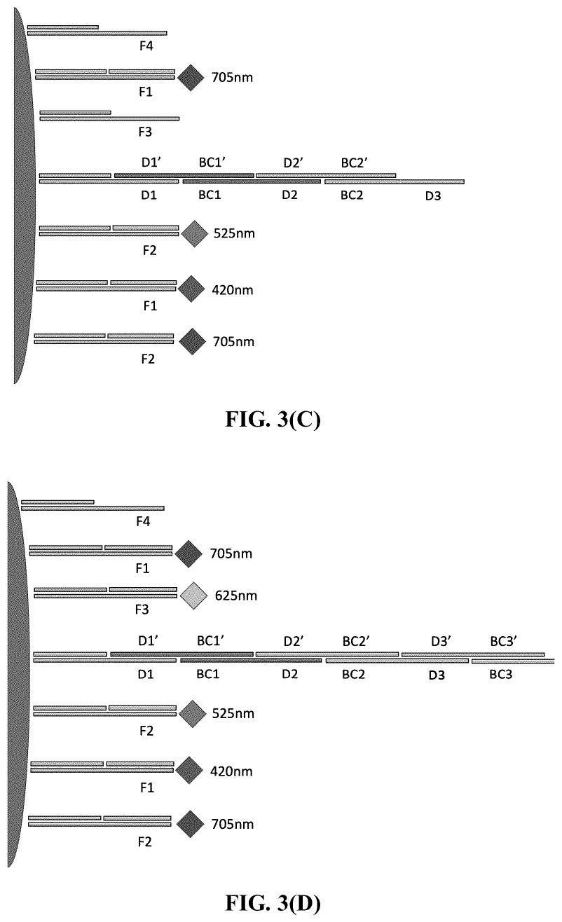

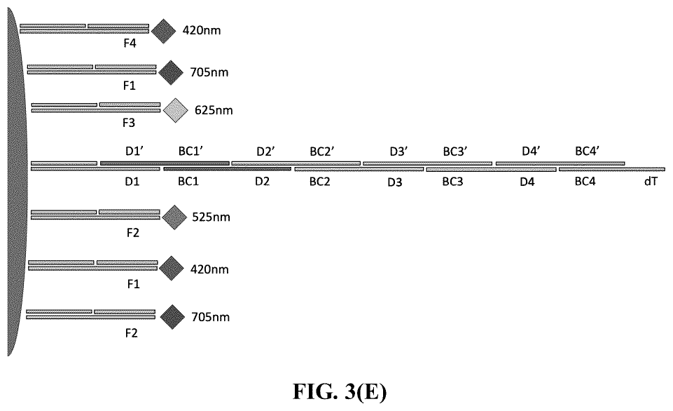

Each bead carries both a fluorescent barcode (as disclosed herein) and DNA barcodes, where the purpose of the fluorescent barcode is to decode a unique DNA barcode on each bead so the data from time lapse images can be correlated to molecular readouts measured by next generation sequencing (NGS). In some non-limiting embodiments, the microwells are sealed with the beads. The microwells can be sealed with beads after the end of live cell imaging. The microwells can be conically shaped so that each microwell traps only a single bead from a population of beads that have an optimal size range. The beads can be used to seal each microwell so that molecular reactions in one microwell are partitioned and kept separate from reactions in adjacent microwells, thus limiting cross-contamination.
Bead encoding scheme—The bead barcodes can comprise a fluorescent barcode and DNA barcode which is synthesized by conjugating beads to a combination of fluorescent moieties and DNA sequences. Each bead can contain one or more identical oligonucleotides which comprise the DNA barcodes. Each bead can also contain a combination of fluorescent moieties that is unique to each bead, and where the fluorescent barcode has a known 1:1 correlation with the DNA barcode sequence. Some examples of fluorescent moieties that can be used for this purpose include, but are not necessarily limited to, organic molecules, quantum dots, metallic nanoparticles, and other nanoscale materials that emit fluorescent light. The fluorescent moieties are attached to the beads through cleavable linkers, which makes it possible to increase the number of fluorescent combinations that can be decoded through a sequence of imaging steps. In some non-limiting embodiments, the cleavable linkers include a DNA sequence having a restriction site that is selectively cleavable by a selected restriction enzyme. The decoding process can involve first obtaining a fluorescent measurement of a bead having one or more fluorescent moieties attached thereto by one or more cleavable linkers, then introducing a cleaving agent to remove a subset of fluorescent moieties from the beads, and then re-imaging the bead to detect the difference of optical signal in one or more fluorescent channels. This process can be repeated sequentially N times, each time cleaving a different linker, and each time using M different fluorescent channels in parallel, such that there are 2\hat{ }(N×M) combinations of different fluorescent barcodes on each bead. As a non-limiting example, it is possible to create 65,536 unique fluorescent barcode combinations by using 4 fluorescent channels (e.g., molecules that emit light at wavelengths of 420 nm, 525 nm, 620 nm, and 705 nm) and 4 different cleavable linkers (e.g. using restriction enzymes that cleave specific DNA sequences), which yields 2\hat{ }(4×4)=65,536 uniquely decodable color combinations.
Bead barcode synthesis procedure—Bead barcodes, comprising a fluorescent barcode and a DNA barcode, are synthesized through a split pool functionalization process that incorporates a process used to prepare bead-based DNA barcodes for single cell RNA-sequencing library preparation workflows. Suitable materials for preparing the DNA barcodes for use with the invention include, but are not necessarily limited to, GEM beads sold by 10X Genomics™, and the Drop-Seq beads sold by LGC Biosearch™ and ChemGenes™. The procedure involves evenly dividing a batch of beads into a number of wells. In this non-limiting example, there are 16 wells. A unique DNA sequence is added to each of the 16 wells, which becomes attached to one common anchor sequence present on all beads. Next, N different fluorescent moieties are added to each well. In this non-limiting example, there are 4 different fluorescent moieties, which are attached to the beads through a common cleavable anchor sequence that is present on all beads. After this first step, each bead will have one of 16 different DNA barcode sequences (i.e., one for each well), and one of 16 different fluorescent combinations. In this example, a fluorescent barcode of 1001 corresponds to beads that became conjugated to two fluorescent moieties, which emits light at 420 nm and 705 nm, respectively. Likewise, a fluorescent barcode of 1110 corresponds to beads that became conjugated to three different fluorescent moieties the emit light at 420 nm, 525 nm, and 625 nm, respectively. After this first barcoding step, the beads from all 16 wells are combined into one tube, mixed, and then evenly divided into 16 separate wells before initiating the next step of the split pool functionalization process. In this second step, a different set of 16 unique DNA sequences is added to the wells (one unique sequence per well), which become attached to the first DNA barcode sequence to form a linear chain of DNA barcodes. At the same time, a set of 4 different fluorescent moieties are again added to each well, which become attached to the beads through a different cleavable anchor sequence that is present on all beads. After this second step, each bead will have one of 16×16=256 unique DNA barcode sequences and one of 16×16=256 unique fluorescent barcodes. This process is iteratively repeated until the desired number of DNA and fluorescent barcode sequences is obtained. For example, if this process is repeated 4 times in the manner described above, it would yield 65,536 unique DNA barcode sequences which are correlated to a known set of 65,536 unique fluorescent barcodes.
Encoding Example. Polystyrene beads with a mean diameter of ˜ 30 μm are functionalized with 5 different DNA sequences, named D1, F1, F2, F3, and F4, as shown in FIG. 3(A). One of these DNA sequences is used to attach the DNA barcode (D1), and the remaining 4 DNA sequences (F1, F2, F3, and F4) are used to attach the fluorescent moieties (one for each step in the split pool process). In the first step, beads are evenly divided into 16 wells, and then a double stranded sequence having a unique barcode sequence and a single strand overhang that is complementary to D1 is added to each well in order to ligate the first section of the DNA barcode to each bead. The DNA sequence also has a different single strand overhang at the other end (D2′) that will be used to attach the next set of DNA barcodes in series via chained ligation. At the same time, a unique combination of quantum dots selected from a set that emits light at 420 nm, 525 nm, 625 nm, and 705 nm is ligated to the F1 sequence on the beads in 16 different combinations (one for each well). In this example, the first fluorescent code is 1001, which means that a mixture of quantum dots that fluoresce at 420 nm and 705 nm are attached to the F1 sequences on the beads, as shown in FIG. 1(B).

The beads are then pooled and split again into 16 wells. In the second step, a double stranded sequence with a single strand overhang that is complementary to D2 is added to each well to ligate the second section of the DNA barcode to the first section of the DNA barcode that was attached in the previous step. The DNA sequence also has a different single strand overhang at the other end (D3′) that will be used to attach the third DNA barcode in series to the second barcode. At the same time, a unique combination of quantum dots selected from a set that emits light at 420 nm, 525 nm, 625 nm, and 705 nm is ligated to the F2 sequence on the beads. In this example, the second fluorescent code is 0101, which means that a mixture of quantum dots that fluoresce at 525 nm and 705 nm are attached to the F2 sequences on the beads, as shown in FIG. 3(C).

The beads are again pooled and split into 16 different wells. In the third step, a double stranded sequence with a single strand overhang that is complementary to D3 is added to each well in order to ligate the third section of the DNA barcode to the second section of the DNA barcode that was attached in the previous step. The DNA sequence also has a different single strand overhang at the other end (D4′) that will be used to attach the fourth DNA barcode in series to the third barcode. At the same time, a unique combination of quantum dots selected from a set that emits light at 420 nm, 525 nm, 625 nm, and 705 nm is ligated to the F3 sequence on the beads. In this example, the third fluorescent code is 0010, which means that quantum dots fluorescing at 625 nm are attached to the F3 sequences on the beads, as shown in FIG. 3(D).

The beads are again pooled and split into 16 different wells. In this last step, a double stranded sequence with a single strand overhang that is complementary to D4 is added to each well in order to ligate the fourth section of the DNA barcode to the third section of the DNA barcode that was attached in the previous step. The fourth section of DNA also has a single stranded capture motif that is used to bind to specific panel of cellular RNA (e.g., polyT tail to capture mRNA). Meanwhile, a unique combination of quantum dots selected from a set that emits light at 420 nm, 525 nm, 625 nm, and 705 nm is ligated to the F4 sequence on the beads. In this example, the fourth fluorescent code is 1000, which means that quantum dots fluorescing at 420 nm are attached to the F4 sequences on the beads, as shown in FIG. 3(E).

After this process is complete, each bead will contain a plurality of DNA barcodes that are identical on each bead, but different between different beads, and wherein the DNA barcode consists of 4 discrete smaller barcode sections arranged in series. Each bead will also contain a unique combination of fluorescent barcodes that is different between different beads, and where the fluorescent barcode is directly correlated to the DNA barcode. Decoding Example: The bead barcodes synthesized through the process described in FIGS. 3(A)-3(E) are placed in the microwells which is size matched to the bead such that only one bead can fit into the microwell. Next, RNA sequencing libraries are prepared directly inside the microwells, which involves the capture of mRNA onto the dT capture tail of the bead, followed by reverse transcription to form cDNA extended off the dT sequence. The decoding process then involves the sequential addition of restriction enzymes to identify the unique sequences on each bead. In this specific example, a first restriction enzyme is added to cut off the quantum dots attached to the F1 anchor sequences. The fluorescent signal on that bead before and after exposure to the restriction enzyme leads to a step change in the bead fluorescence in the 420 nm and 705 nm wavelengths, thus revealing that the first fluorescent code is 1001. Next, a second restriction enzyme is added to cut off the quantum dots attached to the F2 anchor sequences, which leads to a step change reduction in the bead fluorescence in the 525 nm and 725 nm wavelengths, revealing that the second fluorescent code is 0101. This process is then repeated for the F3 anchor sequence, revealing that the third fluorescent code is 0010, and then the F4 anchor sequence, revealing that the fourth fluorescent code is 1000. The full fluorescent barcode of the bead is therefore 1001 0101 0010 1000, which corresponds to a specific DNA barcode sequence (BC1-BC2-BC3-BC4) that can be identified from a lookup table.

Barcode collisions: The barcode diversity of the present assay is sufficient for many single cell RNA-sequencing applications that require profiling of more than a few thousand single cells. For example, in one embodiment we may use one well of a 96-well plate, having 30,000 microwells at the bottom, to capture ˜2,000 single cells. These microwells would then be sealed with beads that carry 65,536 unique DNA barcodes which are correlated to a known set of 65,536 unique fluorescent barcodes. In this example, we expect that less than 1% of microwells containing a single cell would have the same fluorescent and DNA barcode as another microwell that also has one or more single cells. Those microwells that have a barcode collision can be optically detected and removed during bioinformatic data processing steps, ensuring higher confidence in the single cell RNA-seq datasets. Higher throughput single cell RNA-sequencing libraries can be prepared by carrying out the same workflow in all wells of a 96-well plate. At the end of the workflow, a barcoded adapter that is unique to each well (BC5) is attached to the cDNA derived from each bead, thus adding another barcode to each cDNA molecule which becomes BC1-BC2-BC3-BC4-BC5. This approach will enable the processing of hundreds of thousands to millions of single cells in parallel, along with the corresponding time lapse images.

The fluorescent barcoding strategy described in at least this example lends itself to a number of applications. First, it improves data quality in single cell sequencing applications by excluding data from microwells containing more than one cell.

Second, it can be used in antibody discovery to assign antibody-encoding gene sequences to individual microwells in which antibodies are secreted by single B-cells and examined using optical readouts. In this example, small antibody-capture beads and B-cells will be loaded into wells of the microwell arrays. During an incubation period, antibodies secreted by B-cells will bind to beads. The antibody binding affinity will be measured through the addition of fluorescent antigens to the assay media. Binding of antigens to the beads will be detected by fluorescent microscopy to evaluate affinity and specificity of antibodies secreted by B-cells. At the end of the experiment, the beads carrying both a fluorescent and a DNA barcode will be loaded into the microwells, and cells will be lysed to enable the antibody encoding mRNA transcripts to be captured and reverse transcribed onto the beads to form cDNA that is extended off the DNA barcode sequences connected to the bead through D1. The fluorescent barcodes on each bead will be decoded using the decoding procedure in this example in order to identify the DNA barcode sequences of each microwell. Next, the beads will be retrieved from the microwells and the cDNA from the beads will be amplified and prepared for measurement by next generation sequencing (NGS). The data output from the sequencer will consist of a multitude of DNA sequences, some of which will contain DNA barcodes that are unique to a bead, and which also contain unique antibody encoding region associated with the cell that was present in the microwell. The specific microwell associated with each DNA barcode will be determined through the decoding procedure in this example, therefore, it is possible to correlate the results from the fluorescent binding assay with the specific antibody encoding sequences from the same cells in order to identify the antibody encoding sequences of specific cells that produce antibodies with the highest affinity and specificity for a target antigen. Notably, this can be performed in a high throughput manner that greatly exceeds the number of cells that could be individually retrieved from specific microwells using a cell picker or other currently existing image-based sorting methods.

Third, the fluorescent barcoding strategy can be employed for identification of potent T-cell receptors by enabling the assignment of gene sequences encoding T-cell receptors to individual microwells in which cytotoxicity of single T-cells towards target cells will be examined using optical readouts. In this example, T-cells and target cells, such as cancer cells, will be loaded into wells of the microwell arrays to create an array of discrete co-cultures. Growth and death of both cell types will be monitored by time-lapse microscopy to evaluate cytotoxicity of T-cells toward target cells. At the end of the experiment, the beads carrying both a fluorescent and a DNA barcode will be used to seal the microwells, and cells will be lysed to enable the T-cell receptor encoding mRNA transcripts to be captured and reverse transcribed onto the beads to form cDNA that is extended off the DNA barcode sequences connected to the bead through D1. Sealing the microwells in this and other processes disclosed herein can be practiced using the methods, reagents, and devices disclosed in the following publications, the disclosures of which are incorporated herein for all purposes: US Patent Application Publication Nos. 2018/0280918 and 2019/0137481.

The fluorescent barcodes on each bead will be decoded using the decoding procedure in this example in order to identify the DNA barcode sequences of each microwell. Next, the beads will be retrieved from the microwells and the cDNA from the beads will be amplified and prepared for measurement by next generation sequencing (NGS). The data output from the sequencer will consist of a multitude of DNA sequences, some of which will contain DNA barcodes that are unique to bead, and which also contain unique T-cell receptor encoding region associated with the cell that was present in the microwell. The specific microwell associated with each DNA barcode will be determined through the decoding procedure in this example, therefore, it is possible to correlate the specific T-cell receptor encoding sequences with the results from T-cell cytotoxicity assay toward target cells. Notably, this can be performed in a high throughput manner that greatly exceeds the number of cells that could be individually retrieved from specific microwells using a cell picker or other currently existing image-based sorting methods.

Fourth, the fluorescent barcoding strategy can assist in identification of molecular targets by CRISPR screening by assigning CRISPR modification in the genome to individual microwells in which functional assays on these cells, such as cell cycle arrest or formation of focal adhesions, is examined using optical readouts. In this example, CRISPR modified cells will be loaded into wells of the microwell arrays. Functional assay for these cells will be performed using imaging-based readouts, such as cells that proliferate or experience cell cycle arrest in the presence of a drug or perturbation, or cells that express different morphological features. At the end of the experiment, the beads carrying both a fluorescent and a DNA barcode will be loaded into the microwells, and cells will be lysed to enable the mRNA transcripts with CRISPR edits to be captured and reverse transcribed onto the beads to form cDNA that is extended off the DNA barcode sequences connected to the bead through D1. If CRISPR edits are not detectable by transcriptional analyses then the regions of edited genome will be amplified followed by the capture of amplicons (products of amplification) to the bead and extension or ligation of the amplicons to the DNA barcode. The fluorescent barcodes on each bead will be decoded using the decoding procedure in this example to identify the DNA barcode sequences of each microwell. Next, the beads will be retrieved from the microwells and the cDNA from the beads will be amplified and prepared for measurement by next generation sequencing (NGS). The data output from the sequencer will consist of a multitude of DNA sequences, some of which will contain DNA barcodes that are unique to bead, and which also contain unique CRISPR barcodes that are associated with the cell that was present in the microwell. The specific microwell associated with each DNA barcode will be determined through the decoding procedure in this example, therefore, it is possible to correlate phenotypic characteristics of each cell with its CRISPR modification. Notably, this can be performed in a high throughput manner that greatly exceeds the number of cells that could be individually retrieved from specific microwells using a cell picker or other currently existing image-based sorting methods.

Fifth, the fluorescent barcoding strategy can be used to identify drug resistance mechanisms in cancer cells. Optical readout can be used to visualize clonal expansion of cancer cells exposed to different concentrations of drugs, whereas the specific mutations and altered cell signaling will be revealed by single cell sequencing. In this example, cancer cells will be first loaded into wells of the microwell arrays containing different concentrations of drugs. Time lapse images of microwells will be used to determine growth rates and cell death. At the end of the experiment, the beads carrying both a fluorescent and a DNA barcode will be loaded into the microwells, and cells will be lysed to allow mRNA to hybridize to capture oligos for transcriptome analyses using NGS. The fluorescent barcodes on each bead will be decoded using the decoding procedure in this example. The correlation between phenotypic measurements of cell function and transcriptional profiling will be used to identify drug resistance mechanism of cancer cells.

Sixth, the fluorescent barcoding strategy can allow investigation of heterogeneity of cell populations by assigning molecular signatures revealed by single cell transcriptional or other sequencing-based readouts with live-cell imaging readouts in each microwell. In this example, cells will be first loaded into wells of the microwell arrays. Time lapse images of microwells will be used to measure phenotypic parameters of cells. At the end of the experiment, the beads carrying both a fluorescent and a DNA barcode will be loaded into the microwells, and cells will be lysed to allow mRNA to hybridize to capture oligos for transcriptome analyses using NGS. The fluorescent barcodes on each bead will be decoded using the decoding procedure in this example. The correlation between phenotypic measurements of cell function and transcriptional profiling will be used to identify molecular mechanism responsible for differences detected by live cell imaging.

### Example 2

Generation of Fluorescent Barcoded Beads with Attached Chemical Compounds for Optical Demultiplexing by Multi-Color Fluorescence Imaging for Drug Screening
Basic workflow. The overall goal is to measure phenotypic information from cells exposed to chemicals. Experiments are performed in optically clear microwell arrays, which are fabricated by attaching an embossed microwell array film to the bottom of a 96-well plate frame. Beads with attached chemical molecules are loaded into one or more of the wells of the 96-well plate microwells. The beads sediment into the microwells to the bottom of each well. Each bead carries a fluorescent barcode, where the purpose of the fluorescent barcode is to encode the chemical on the bead. Decoding of the fluorescent barcodes is performed to map distribution of the chemicals in the microwells. At the end of the decoding, cells are loaded into wells of the 96-well plate containing the beads. The cells sediment into the microwells to the bottom of each well, thus creating thousands of discrete microwell-based cell cultures. The chemical is released from these beads. Standard brightfield and epi-fluorescent imaging is used to collect phenotypic information, such as the number of cells in each microwell, activation of a reporter, or interaction between different types of cells. In some cases, it is desirable to also quantify cellular secretions in each well by co-assembling beads into the microwells that have capture moieties for different molecules, including but not limited to, nucleic acids, antibodies, cytokines, or chemokines. The molecules captured on the bead are measured using image-based readouts. One non-limiting embodiment of this application is to find specific compounds that hinder the growth rate of cancer cells by performing random placement of chemical compounds in high throughput microwell-based assays.
One-Bead-One-Compound (OBOC) encoding scheme—Each bead contains a plurality of identical chemicals linked to the bead via a cleavable linker. Each bead also contains a combination of fluorescent moieties that is unique to each bead, where the fluorescent barcode has a known 1:1 correlation with the chemical composition. Some non-limiting examples of fluorescent moieties that can be used for this purpose include, but are not necessarily limited to, organic molecules, quantum dots, metallic nanoparticles, other nanoscale materials that emit fluorescent light, or combinations thereof. The fluorescent moieties are attached to the beads through cleavable linkers, which makes it possible to increase the number of fluorescent combinations that can be decoded through a sequence of imaging steps. The decoding process involves first obtaining a fluorescent measurement of a bead, then introducing a cleaving agent to remove a subset of fluorescent moieties from the beads, and then re-imaging the bead to detect the difference of optical signal in one or more fluorescent channels. This process can be repeated sequentially N times, each time cleaving a different linker, and each time using M different fluorescent channels in parallel, such that there are 2\hat{ }(N×M) combinations of different fluorescent barcodes on each bead. As a non-limiting example, it is possible to create 65,536 unique fluorescent barcode combinations by using 4 fluorescent channels (e.g., molecules that emit light at wavelengths of 420 nm, 525 nm, 620 nm, and 705 nm) and 4 different cleavable linkers (e.g. using restriction enzymes that cleave specific DNA sequences), which yields 2\hat{ }16=65,536 uniquely decodable color combinations.
OBOC synthesis procedure—Chemicals and fluorescent barcodes are synthesized through a split pool functionalization process. The procedure involves evenly dividing a batch of beads into several wells, such as 16 wells in this non-limiting example. A unique chemical block is added to each of the 16 wells, which becomes attached to one common cleavable anchor sequence that is present on all beads. Next, N different fluorescent moieties are added to each well. For this non-limiting example, 4 different fluorescent moieties are used, which become attached to the beads through a common cleavable anchor sequence that is present on all beads. After this first step, each bead will have one of 16 different chemical blocks (i.e., one for each well), and one of 16 different fluorescent combinations. After this first barcoding step, the beads from all 16 wells are combined into one tube, mixed, and then evenly divided into 16 separate tubes before initiating the next step of the split pool functionalization process. In this second step, a different set of 16 unique chemical blocks is added to the wells (one unique chemical per well), which become attached to the first chemical block. At the same time, a set of 4 different fluorescent moieties are again added to each well, which become attached to the beads through a different cleavable anchor sequence that is present on all beads. After this second step, each bead will have one of 16×16=256 unique chemicals and one of 16×16=256 unique fluorescent barcodes. This process is iteratively repeated until the desired number of chemicals and fluorescent barcode sequences is obtained. For example, if this process is repeated 4 times in the manner described above, it would yield 65,536 unique chemicals which are correlated to a known set of 65,536 unique fluorescent barcodes.

The fluorescent encoding strategy in this example can be used to encode a chemical compound on a bead. In this application, chemicals composed of three or more blocks will be assembled in split-pull synthesis. These chemicals will be linked to beads via a cleavable linker. Beads will be seeded to microwells and chemicals present in each microwell identified. Reporter cells will be loaded followed by release of the chemical from these beads. Optical readout will be used to detect the signal from reporter cells.

### Example 3

Generation of Fluorescent Barcoded Beads with Absorbed Chemical Compounds for Optical Demultiplexing by Multi-Color Fluorescence Imaging for Drug Screening

Basic workflow. The overall goal is to measure the responses of cells to chemicals. Experiments are performed in optically clear microwell arrays, which are fabricated by attaching an embossed microwell array film to the bottom of a 96-well plate frame. Cells are loaded into one or more wells of the 96-well plate. The cells sediment into the microwells to the bottom of each well, thus creating thousands of discrete microwell-based cell cultures. Next, beads with absorbed chemical molecules are loaded and seal the microwells. The microwells are designed to be conically shaped so that each microwell traps only a single bead from a population of beads that have an optimal size range. The bead is used to seal each microwell so that chemicals released from the beads are concentrated in the microwells with minimal cross-contamination to adjacent microwells. Standard brightfield and epi-fluorescent imaging is used to collect phenotypic information, such as the number of cells in each microwell, activation of the reporter, or interaction between different types of cells. One non-limiting use for this embodiment of the invention is to find specific compounds or combinations of compounds that hinder the growth rate of cancer cells by performing random placement of chemical compounds in high throughput microwell based assays.

Each bead carries a fluorescent barcode, where the purpose of the fluorescent barcode is to decode a chemical absorbed on each bead. Each bead might also have oligos with a unique DNA barcode to capture mRNA or other molecules from a cell or cells in the microwells for next generation sequencing (NGS). At the end of the live cell experiment, optical decoding is performed to map distribution of the chemicals in the microwells in order to correlate the effect of specific compounds to time lapse images of individual microwell based cultures. Optionally, the fluorescent barcode can also decode a unique DNA barcode on each bead, so the chemical identity is correlated with molecular readouts measured by NGS.
Fluorescent encoding scheme—Each group of beads contains a combination of fluorescent moieties that is unique to each group, where the fluorescent barcode has a known 1:1 correlation with the chemical absorbed by the beads. Optionally, the fluorescent barcode also encodes a DNA barcode that is attached to the beads. Some examples of fluorescent moieties that can be used for this purpose include, but are not necessarily limited to, organic molecules, quantum dots, metallic nanoparticles, other nanoscale materials that emit fluorescent light, or combinations thereof. The fluorescent moieties are attached to the beads through cleavable linkers, which makes it possible to increase the number of fluorescent combinations that can be decoded through a sequence of imaging steps. The decoding process involves first obtaining a fluorescent measurement of a bead, then introducing a cleaving agent to remove a subset of fluorescent moieties from the beads, and then re-imaging the bead to detect the difference of optical signal in one or more fluorescent channels. This process can be repeated sequentially N times, each time cleaving a different linker, and each time using M different fluorescent channels in parallel, such that there are 2\hat{ }(N×M) combinations of different fluorescent barcodes on each bead. As a non-limiting example, it is possible to create 65,536 unique fluorescent barcode combinations by using 4 fluorescent channels (e.g., molecules that emit light at wavelengths of 420 nm, 525 nm, 620 nm, and 705 nm) and 4 different cleavable linkers (e.g. using restriction enzymes that cleave specific DNA sequences), which yields 2\hat{ }16=65,536 uniquely decodable color combinations.
Fluorescent barcode synthesis procedure—Fluorescent, and optionally DNA, barcodes are synthesized through a split functionalization process. The procedure involves evenly dividing a batch of beads into several wells. In this non-limiting example, there are 16 wells. N different fluorescent moieties are added to each well. In this non-limiting example, there are 4 different fluorescent moieties which become attached to the beads through a common cleavable anchor sequence that is present on all the beads. An optional well-specific DNA block is added to each of the 16 wells, which becomes attached to one common anchor present on all beads. After this first step, each bead will have one of 16 different fluorescent combinations and, optionally, one of 16 different DNA blocks (i.e., one for each well). After this first barcoding step, the beads from each well are evenly split into 16 separate wells before initiating the next step of the functionalization process. In this second step, a set of 4 different fluorescent moieties are again added to each well, which become attached to the beads through a different cleavable anchor sequence that is present on all the beads. Optionally, a unique DNA block is added together with each fluorescent code combination to the wells which is then attached to the first DNA block. After this second step, there will be 16×16=256 wells containing a unique fluorescent barcode, and optionally the same number of unique DNA, barcodes. This process is iteratively repeated until the desired number of DNA and fluorescent barcode sequences is obtained.

In this example, cells will be first loaded into wells of the microwell arrays. Beads will be seeded into the microwells and the compound that is physiosorbed into the beads will elute out of the beads into microwells. Time lapse images of microwells will be used to determine phenotypic parameters of cell function, such as growth rates and measurement of other metabolic processes. Optionally, at the end of the live cell experiments, cells will be lysed to allow mRNA to hybridize to optional capture oligos for NGS analyses. The fluorescent barcodes on each bead will be decoded using the decoding procedure in this example. The correlation between phenotypic measurements of cell function, optional transcriptional profiling, and the identification of the specific compound in each microwell will be used to identify hits in a high throughput manner.

1. A method of making a library of fluorescently barcoded microparticles, comprising:
a) providing microparticles;
b) linking one or more fluorophores to said microparticles by one or more cleavable linkers thereby providing encoded microparticles having a plurality of fluorescent barcodes.

2. The method of claim 1, wherein said one or more fluorophores comprises a plurality of fluorophores that are different from one another.

3. The method of claim 1, wherein said one or more cleavable linkers comprise a plurality of cleavable linkers that are different from one another.

4. The method of claim 2, wherein said plurality of fluorophores includes four or more fluorophores that are different from one another.

5. The method of claim 3, wherein said plurality of cleavable linkers includes four or more cleavable linkers that are different from one another.

6. The method of claim 1, further comprising attaching one or more tags to said encoded microparticles.

7. The method of claim 6, wherein said one or more tags are selected from an oligonucleotide, an antibody, a protein, a peptide, a small molecule, and combinations thereof.

8. The method of claim 6, wherein said one or more tags are associated with said plurality of fluorescent barcodes.

9. A method of color encoding DNA barcodes on microparticles, comprising:
a) providing a microparticle;
b) performing an attaching step wherein a first oligonucleotide barcode is attached to said microparticle;
c) performing a first color encoding step wherein a first one or more fluorophores is linked to said microparticle by a first one or more cleavable linkers, wherein the combination of said first one or more fluorophores and said first one or more cleavable linkers provide said microparticle with a first fluorescent barcode that identifies said first oligonucleotide barcode.

10. The method of claim 9, further comprising (i) ligating a second oligonucleotide barcode to said first oligonucleotide barcode, and (ii) performing a second color encoding step wherein a second one or more fluorophores is linked to said microparticle by a second one or more cleavable linkers, wherein the combination of said second one or more fluorophores and said second one or more cleavable linkers provide said microparticle with a second fluorescent barcode that identifies said second oligonucleotide barcode.

11. The method of claim 10, further comprising (i) ligating a third oligonucleotide barcode to said second oligonucleotide barcode, and (ii) performing a third color encoding step wherein a third one or more fluorophores is linked to said microparticle by a third one or more cleavable linkers, wherein the combination of said third one or more fluorophores and said third one or more cleavable linkers provide said microparticle with a third fluorescent barcode that identifies said third oligonucleotide barcode.

12. The method of claim 9, wherein said first one or more fluorophores, said second one or more fluorophores, and said third one or more fluorophores are different from one another.

13. The method of claim 9, wherein said first one or more cleavable linkers, said second one or more cleavable linkers, and said third one or more cleavable linkers are different from one another.

14. A method of identifying a microparticle using a fluorescent barcode, comprising:
a) providing a microparticle having one or more fluorophores linked to said microparticle by one or more cleavable linkers, wherein the combination of said one or more fluorophores and said one or more cleavable linkers provides said microparticle with an identifying barcode;
b) contacting said microparticle with at least one cleaving agent to release at least one of said one or more fluorophores from said microparticle;
c) detecting said release of said at least one fluorophore;
d) correlating said release of said at least one fluorophore with said one or more cleavable markers thereby identifying said microparticle as having said identifying barcode.

15. The method of claim 14, wherein contacting step 14(b) and detecting step 14(c) are repeated at least once prior to correlating step 14(d).

16. The method of claim 14, wherein said one or more cleavable linkers are different from one another, and contacting step 14(b) is repeated with different cleaving agents.

17. The method of claim 14, further comprising an initial detection step wherein said one or more fluorophores are detected while linked to said microparticle prior to contacting step 14(b); and detecting step 14(c) comprises comparing said one or more fluorophores on said microparticles from said initial detection step to said one or more fluorophores on said microparticles after said contacting step 14(b).

18. The method of claim 14, wherein said microparticle comprises one or more tags that are associated with said identifying barcode, and said one or more tags are selected from an oligonucleotide, an antibody, a protein, a peptide, a small molecule, and combinations thereof.

19. The method of claim 18, wherein correlating step 14(d) identifies said microparticle as having said one or more tags.

20-63. (canceled)
